(19) United States
(12) Patent Application Publication
(10) Pub. No.: US 2026/0182853 A1
(43) Pub. Date: Jul. 2, 2026

(54) VIBRATION DETECTION DEVICE AND VIBRATION DETECTION METHOD

(71) Applicant: Mitsubishi Electric Corporation, Tokyo (JP)

(72) Inventors: Saki WADA, Tokyo (JP); Shintaro IZUMI, Kobe-shi (JP); Kengo NISHIMOTO, Tokyo (JP)

(73) Assignee: Mitsubishi Electric Corporation, Tokyo (JP)

(21) Appl. No.: 19/545,972

(22) Filed: Feb. 20, 2026

Related U.S. Application Data
(63) Continuation of application No. PCT/JP2023/036662, filed on Oct. 10, 2023.

(51) Int. Cl. A61B 5/05; A61B 5/00; A61B 5/11; A61B 5/113

(52) U.S. Cl. CPC A61B 5/05; A61B 5/1102; A61B 5/1126; A61B 5/113; A61B 5/7267; A61B 2562/0219; A61B 2562/06

(57) ABSTRACT

A vibration detection device includes: a radio wave sensor to detect a change in near field electromagnetic field; an inertial sensor; and a signal processing device to input waveform data of the radio wave sensor and waveform data of the inertial sensor to a learning model, and acquire from the learning model an inference result of vibration inferred by the learning model.

### CROSS REFERENCE TO RELATED APPLICATION

This application is a Continuation of PCT International Application No. PCT/JP2023/036662, filed on October 10, 2023, which is hereby expressly incorporated by reference into the present application.

### TECHNICAL FIELD

The present disclosure relates to a vibration detection technique.

### BACKGROUND ART

Patent Literature 1 discloses a technique related to a pulse wave information processing device that processes pulse wave information indicating a pulse wave of a passenger of a mobile body, and includes an information acquisition unit that can acquire first pulse wave information acquired by a first pulse wave information acquisition device, and second pulse wave information acquired by a second pulse wave information acquisition device using a method different from that of the first pulse wave information acquisition device together with reliability degree information indicating reliability of the second pulse wave information, and an information selection unit that selects which one of the first pulse wave information and the second pulse wave information to output as pulse wave information of the passenger on the basis of the reliability degree information.

### CITATION LIST

### PATENT LITERATURE

Patent Literature 1: JP 2020-103461 A

### SUMMARY OF INVENTION

### TECHNICAL PROBLEM

According to the technique of Patent Literature 1, one of the first pulse wave information and the second pulse wave information is alternatively selected as the pulse wave information of the passenger on the basis of the reliability degree information indicating reliability of the second pulse wave information, and therefore there is a problem that, if there is data missing in the selected first pulse wave information or second pulse wave information, measurement accuracy lowers.

The present disclosure has been made on an occasion of recognition of such a problem, and an object of one aspect of the present disclosure is to provide a vibration detection technique that can suppress measurement accuracy from lowering due to data missing.

### SOLUTION TO PROBLEM

One aspect of a vibration detection device according to the embodiment of the present disclosure includes: a radio wave sensor to detect a change in near field electromagnetic field; an inertial sensor; and a signal processor to input waveform data of the radio wave sensor and waveform data of the inertial sensor to a learning model, and acquire from the learning model an inference result of vibration inferred by the learning model, wherein the radio wave sensor detects the change in the electromagnetic field caused by a motion of a lung of a person that is a detection target, the inertial sensor detects local vibration caused by the motion of the lung, and the signal processor infers a peak of respiration of the person as the vibration using all information obtained by the radio wave sensor and the inertial sensor.

### ADVANTAGEOUS EFFECTS OF INVENTION

A vibration detection device according to the embodiment of the present disclosure can suppress measurement accuracy from lowering due to data missing.

### BRIEF DESCRIPTION OF DRAWINGS

FIG. 1A is a configuration diagram of a vibration detection device.

FIG. 1B is a flowchart illustrating an operation example of the vibration detection device.

FIG. 2 is a configuration diagram of a vibration detection device.

FIG. 3A is a view illustrating an example of a loop antenna.

FIG. 3B is a view illustrating an example of a meander dipole antenna.

FIG. 4 is a configuration diagram of a signal processing device.

FIG. 5 is a configuration diagram of a vibration detection device.

FIG. 6 is a configuration diagram of a vibration detection device.

FIG. 7 is a configuration diagram of a signal processing device.

FIG. 8 is a flowchart of adaptive control of a matching circuit performed by the vibration detection device.

FIG. 9 is a configuration diagram of a vibration detection device.

FIG. 10 is a view illustrating a waveform of the vibration detection device and an electrocardiograph.

FIG. 11 is a view illustrating a comparison result obtained by comparing per subject a value of a Root Mean Square Error (RMSE) between a peak interval of a heartbeat waveform inferred using a Conditional Variational AutoEncoder (CVAE), and a peak interval of an electrocardiogram waveform that is a correct answer value.

### DESCRIPTION OF EMBODIMENTS

Various embodiments of the present disclosure will be described in detail below with reference to the accompanying drawings. Note that identical or similar portions are assigned the identical or similar reference numerals in the drawings, and redundant description of these portions will be omitted. Furthermore, a term “or” in the present disclosure is used to mean an inclusive logical OR unless specified in particular.

### Embodiment 1

### <Configuration>

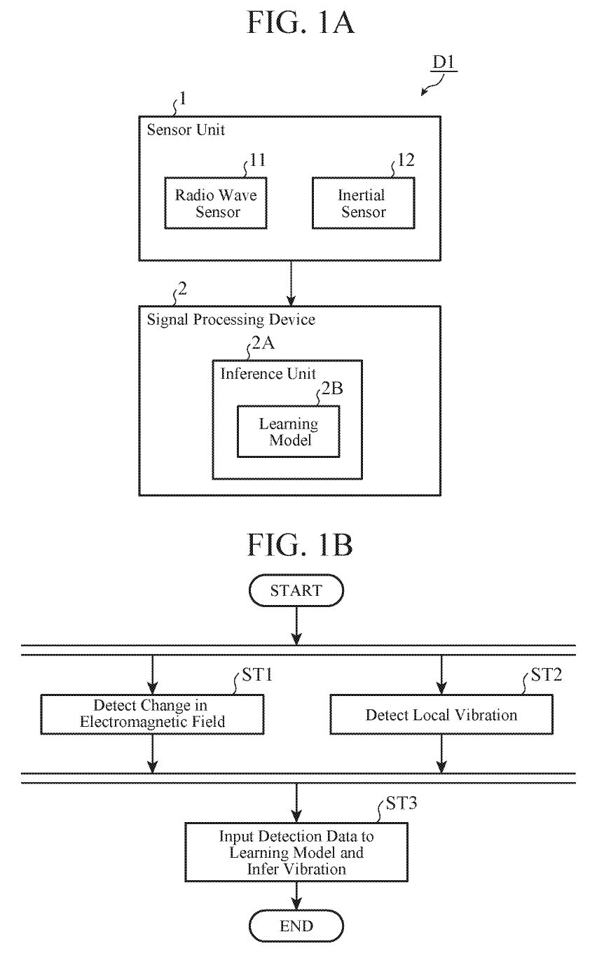

A configuration of a vibration detection device D1 according to Embodiment 1 will be described with reference FIG. 1A. FIG. 1A is a configuration diagram illustrating the vibration detection device D1 according to Embodiment 1. As illustrated in FIG. 1A, the vibration detection device D1 includes a sensor unit 1 and a signal processing device 2.

The sensor unit 1 includes a radio wave sensor 11 and an inertial sensor 12. The radio wave sensor 11 is a near field electromagnetic field type radio wave sensor that uses a small antenna whose wavelength is smaller than a wavelength used by the radio wave sensor 11, and detects a change in near field electromagnetic field of the radio wave sensor 11. A small antenna of low radiation efficiency is used to detect only a change in near field electromagnetic field of the radio wave sensor 11. In the present disclosure, the “near field” means an inner side of a range of a sphere whose distance from the radio wave sensor 11 is λ/2π (λ: wavelength) or less.

By disposing the radio wave sensor 11 near a detection target, the radio wave sensor 11 can observe a motion of the detection target. Here, the “detection target” means an arbitrary object positioned near the radio wave sensor 11. Although typical examples of the detection target include a human lung, a human heart, or part of a device, an object such as a lung of an animal other than a person may be a detection target. A case is assumed where the detection target is, for example, a human heart. When the heart moves, a body surface of the person is fluctuated by the motion of the heart. The fluctuation of this human body surface changes the electromagnetic field. Accordingly, by disposing the radio wave sensor 11 near the human body surface, it is possible to observe the motion of the human heart.

The inertial sensor 12 is a sensor that detects an inertial motion. Examples of the inertial sensor 12 include a triaxial acceleration sensor or a gyro sensor. By disposing the inertial sensor 12 near a detection target, the inertial sensor 12 can detect local vibration caused by the motion of the detection target. The local vibration means vibration at a position at which the inertial sensor 12 is disposed.

The signal processing device 2 includes an inference unit 2A and a learning model 2B. A configuration example of hardware of the signal processing device 2 will be described in Embodiment 2. The learning model 2B is a learning model that learns in advance a correlation between information obtained by the radio wave sensor 11 and the inertial sensor 12 and a peak of a waveform that is a desired signal. A detailed example of the learning model 2B will be described later in Embodiment 5.

The sensor unit 1 is disposed near, for example, a detection target such as a human lung or heart to use. By disposing the sensor unit 1 as described above, it is possible to more accurately detect a change in electromagnetic field or local vibration caused by the motion of the detection target.

### <Operation>

Next, an operation of the vibration detection device D1 will be described with reference FIG. 1B. FIG. 1B is a flowchart illustrating the operation of the vibration detection device D1. In step ST1, the radio wave sensor 11 detects the change in near field electromagnetic field of the radio wave sensor 11. By disposing the radio wave sensor 11 near the detection target, the radio wave sensor 11 detects the change in electromagnetic field caused by the motion of the detection target. It is possible to acquire a change in volume of the detection target that the radio wave sensor 11 approaches in a non-contact manner on the basis of the change in near field electromagnetic field of the radio wave sensor 11.

In step ST2 performed in parallel to step ST1, the inertial sensor 12 acquires local vibration caused by the motion of the detection target in a non-contact manner.

Note that the “non-contact manner” described herein means that the radio wave sensor 11 and the inertial sensor 12 are not in contact with human skin or a device.

By combining sensors of the radio wave sensor 11 and the inertial sensor 12 whose acquisition principles are different, and acquiring a signal that a single sensor cannot acquire, it is possible to complement accuracy deterioration due to data missing.

In step ST3, the inference unit 2A of the signal processing device 2 inputs the information obtained by the radio wave sensor 11 and the inertial sensor 12 to the learning model 2B, and acquires vibration (waveform) of the desired signal as an inference result from the learning model 2B. The inference result includes a peak of the waveform. As described above, data of the two sensors is input to the learning model 2B to infer the vibration, so that, even when there is data missing in one sensor output, it is possible to complement the accuracy deterioration due to data missing and improve an S/N ratio of the desired signal by inferring vibration using data of the other sensor.

The learning model 2B may be trained using a correct answer value in advance or may be sequentially trained using obtained data. Here, the correct answer value refers to, for example, information with secured accuracy such as an electrocardiogram, for example, when training a peak of a heartbeat waveform.

By using the peak of the acquired waveform that is the desired signal as a feature amount and acquired data as the number of channels, a plurality of items of chronological data may be read and inferred. The inference is not limited to algorithms, and may be an algorithm of one of supervised learning, semi-supervised learning, and reinforcement learning as long as the algorithms enable the above operation.

As described above, the vibration detection device D1 includes the sensor unit 1 and the signal processing device 2, and the learning model 2B of the inference unit 2A included in the signal processing device 2 infers a peak of a waveform using all information obtained by the radio wave sensor 11 and the inertial sensor 12 included in the sensor unit 1, so that it is possible to obtain an effect that it is possible to provide a vibration detection device that can complement accuracy deterioration due to data missing and extract a peak of vibration as a feature amount, and has a high S/N ratio.

### Embodiment 2

### <Configuration>

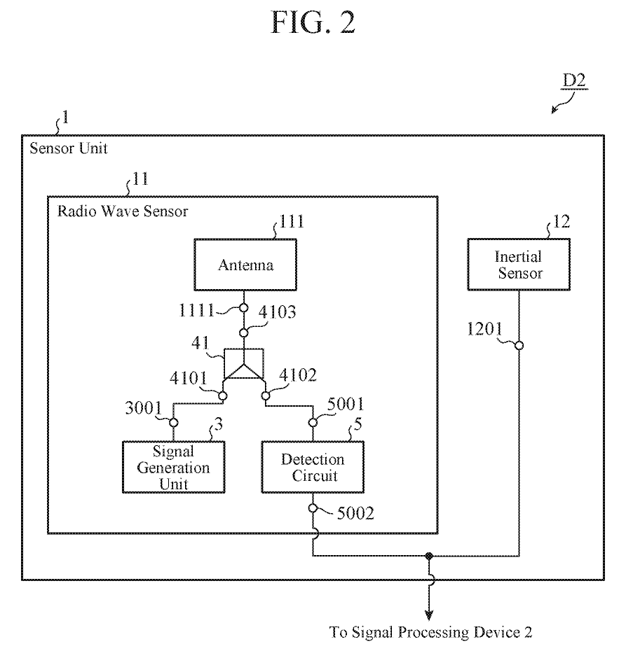

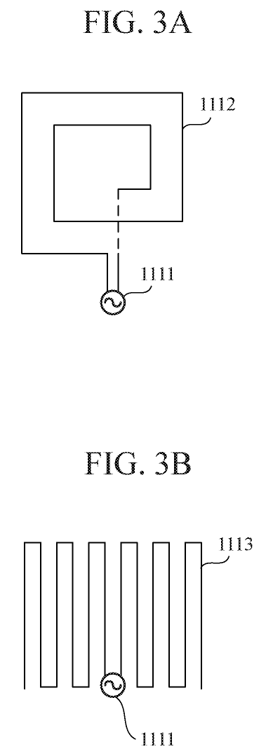

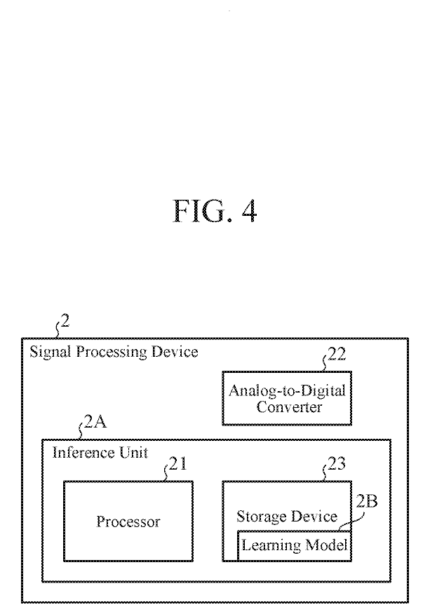

The configuration of the vibration detection device D1 has been described in Embodiment 1. A vibration detection device D2 that is a more specific aspect of the vibration detection device D1 will be described as Embodiment 2 with reference FIGS. 2 to 4. FIG. 2 is a configuration diagram illustrating the vibration detection device D2 according to Embodiment 2. FIGS. 3A and 3B illustrate examples of antennas used for the radio wave sensor, and FIG. 4 is a configuration diagram of a signal processing device. Note that the same reference numerals in FIGS. 2, 3A, 3B, and 4 as those in FIG. 1 indicate identical or corresponding portions.

As illustrated in FIG. 2, the radio wave sensor 11 includes an antenna 111, a signal generation unit 3, a divider circuit 41, and a detection circuit 5, and acquires vibration of a target to be detected using a radio wave in a non-contact manner.

The antenna 111 is a small antenna whose wavelength is smaller than a use wavelength, and a loop antenna or a dipole antenna may be used as the type of the antenna 111. As illustrated in FIG. 2, the antenna 111 includes a terminal 1111.

FIG. 3A illustrates the example of a twin loop antenna 1112, and FIG. 3B illustrates the example a meander dipole antenna 1113. In FIGS. 3A and 3B, the antenna 1112 or 1113 is connected to the terminal 1111. In a case where a wavelength of a use frequency is λ, the length of one side of a rectangular shape of an outermost shape surrounding a conductive medium constituting the antenna is λ/10 or less in any case.

The inertial sensor 12 is a sensor that detects an inertial motion, and includes an output terminal 1201 as illustrated in FIG. 2.

The signal generation unit 3 is a circuit that generates a high frequency signal, and includes an output terminal 3001. For example, the signal generation unit 3 generates the high frequency signal that is a continuous wave signal.

The divider circuit 41 is a high frequency circuit that includes three terminals 4101, 4102, and 4103, and divides a signal input from the terminal 4103 to the terminal 4101 and the terminal 4102. Furthermore, since the terminal 4101 and the terminal 4102 are isolated from each other, the signal input to the terminal 4101 is not output from the terminal 4102, and is output only from the terminal 4103. Note that, since the output terminals only need to be isolated in the divider circuit 41, the divider circuit 41 may be configured using a Wilkinson type divider or a directional coupler whose terminating resistance is connected with an isolation terminal.

The detection circuit 5 includes two terminals 5001 and 5002, and detects a signal input to the input terminal 5001, generates a detection signal, and outputs the detection signal to the output terminal 5002. For the detection circuit, a quadrature detection circuit or the like can be used. In this case, two input terminals and two output terminals need to be prepared, and an I (In Phase) signal and a Q (Quadrature) signal are generated and output as the detection signals. The Q signal is a signal whose phase differs by 90 degrees from that of the I signal.

As illustrated in FIG. 4, the signal processing device 2 includes an analog-to-digital converter 22 that converts an analog signal output from the detection circuit 5 and an analog signal output from the inertial sensor 12 into digital signals, a storage device 23 that stores values of the digital signals converted by the analog-to-digital converter 22, and a processor 21 that controls a circuit or infers data.

The analog-to-digital converter 22 converts the analog signal (detection signal) output from the detection circuit 5 and the analog signal (detection signal) output from the inertial sensor 12 into the digital signals that the processor 21 can control.

The storage device 23 is a generic term of a memory such as a Read Only Memory (ROM) or a Random Access Memory (RAM), and an external storage device such as a hard disk. A program or data used by the processor 21 is stored in the storage device 23, and the processor 21 reads the program stored in the storage device 23 to perform various operations including input/output of data to and from the learning model 2B. Furthermore, the storage device 23 is used as a temporary data storage destination. Furthermore, the signal output from the detection circuit 5 and the signal output from the inertial sensor 12 are also read from and written in the storage device 23.

Next, connection of each terminal will be described. The terminal 1111 of the antenna 111 is connected with the terminal 4103 of the divider circuit 41, the output terminal 3001 of the signal generation unit 3 and the terminal 4101 of the divider circuit 41 are connected, and the terminal 5001 of the detection circuit 5 and the terminal 4102 of the divider circuit 41 are connected.

### <Operation>

Next, an operation of the vibration detection device D2 will be described. First, a signal output from the output terminal 3001 of the signal generation unit 3 is input to the terminal 4101 of the divider circuit 41, and is output from the terminal 4103. The signal output from the terminal 4103 is input to the terminal 1111 of the antenna 111, and is radiated as a radio wave from the antenna 111. The radio wave radiated from the antenna 111 is reflected by the detection target or an object adjacent to the detection target, and input as a signal of a reflected wave to the antenna 111. The reflected signal input from the antenna 111 is output from the terminal 1111, and is input to the terminal 4103 of the divider circuit 41. The reflected signal input to the terminal 4103 of the divider circuit 41 is output from the terminal 4102 of the divider circuit 41, is input to the terminal 5001 of the detection circuit 5, and is output as a detection signal from the terminal 5002 to the signal processing device 2.

Next, when detecting a signal of vibration of a portion caused by the motion of the detection target, the inertial sensor 12 installed near the detection target outputs the detected detection signal from the output terminal 1201 of the inertial sensor 12 to the signal processing device 2.

The signal processing device 2 converts in the analog-to-digital converter 22 signal data acquired by the antenna 111 and the inertial sensor 12 from analog signals into digital signals acceptable by the processor 22, and records signals converted into digital signals and data calculated by the processor 21, in the storage device 23 that has a function of temporarily or semi-permanently storing the signals and the data.

Furthermore, parts that can be mounted on a substrate and have each function described in the present embodiment constitute the vibration detection device D2, so that all functions of the vibration detection device D2 can be implemented on the substrate. For example, the antenna 111 can be formed of a copper foil pattern or can be constituted by parts that can be mounted on the substrate and have the functions of the inertial sensor 12, the signal generation unit 3, the divider circuit 41, the detection circuit 5, and the signal processing device 2. At this time, a permittivity and a layer configuration of the substrate are design matters as appropriate, and any material and layer configuration may be used as long as the material or the layer configuration have the functions of the vibration detection device D2.

As described above, the vibration detection device D2 includes the sensor unit 1 including the radio wave sensor 11 and the inertial sensor 12, and the signal processing device 2. The radio wave sensor 11 includes the antenna 111, the signal generation unit 3, the divider circuit 41, and the detection circuit 5. The signal processing device 2 includes the processor 21, the analog-to-digital converter 22, and the storage device 23. Similarly to Embodiment 1, the learning model 2B of the signal processing device 2 infers a peak of a waveform using all information obtained by the radio wave sensor 11 and the inertial sensor 12 included in the sensor unit 1, so that it is possible to complement accuracy deterioration due to data missing, and implement the vibration detection device D2 that can operate with a high S/N ratio in a compact and low cost manner.

### Embodiment 3

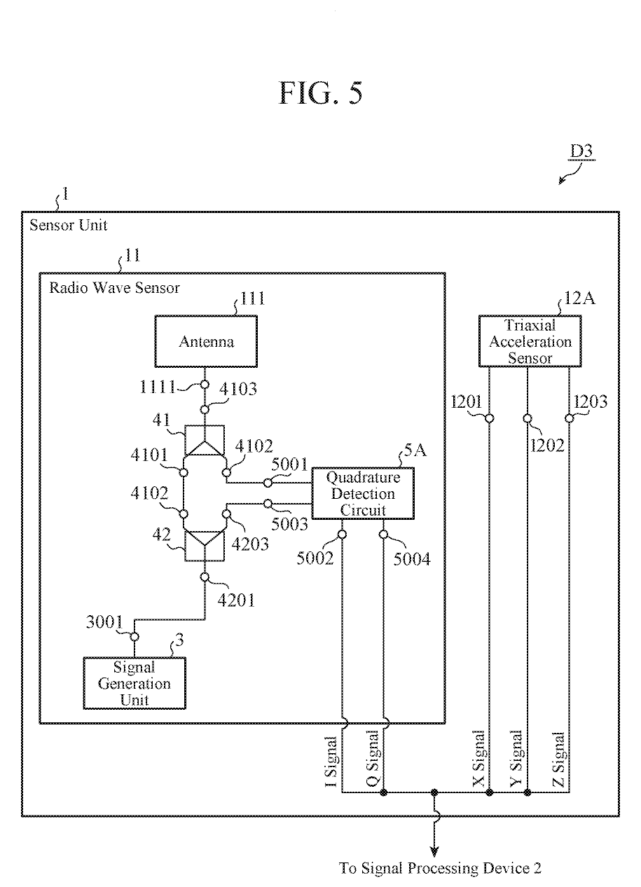

A configuration where a quadrature detection circuit is used as the detection circuit 5 in Embodiment 2, and a triaxial acceleration sensor is used as the inertial sensor 12 will be described in Embodiment 3. FIG. 5 is a configuration diagram illustrating a vibration detection device D3 according to the present embodiment. Note that the same reference numerals in FIG. 5 as those in FIG. 1 indicate identical or corresponding portions.

As illustrated in FIG. 5, the radio wave sensor 11 of the vibration detection device D3 includes the antenna 111, the signal generation unit 3, the divider circuits 41 and 42, and a quadrature detection circuit 5A, and acquires vibration caused by a motion of a detection target using a radio wave in a non-contact manner.

A triaxial acceleration sensor 12A detects an inertial motion along three X, Y, and Z axes. The triaxial acceleration sensor 12A includes three output terminals 1201, 1202, and 1203. The triaxial acceleration sensor 12A outputs an X signal that is a detection signal of the X axis to the output terminal 1201, outputs a Y signal that is a detection signal of the Y axis to the output terminal 1202, and outputs a Z signal that is a detection signal of the Z axis to the output terminal 1203.

The divider circuit 42 is a high frequency circuit that includes three terminals 4201, 4202, and 4203, and divides a signal input from the terminal 4201 to the terminal 4202 and the terminal 4203. Furthermore, since the terminal 4202 and the terminal 4203 are isolated from each other, the signal input to the terminal 4202 is not output from the terminal 4203, and is output only from the terminal 4201.

Note that, since the output terminals in the divider circuit 42 only need to be isolated, the divider circuit 42 may be configured using a Wilkinson type divider or a directional coupler whose terminating resistance is connected with an isolation terminal.

The quadrature detection circuit 5A includes two input terminals (the input terminal 5001 and an input terminal 5003) and two output terminals (the output terminal 5002 and an output terminal 5004), and performs quadrature detection on a signal input to the input terminal 5001 using a local signal input to the input terminal 5003, outputs an I (In Phase) signal from the output terminal 5002, and outputs a Q (Quadrature) signal whose phase differs by 90 degrees from that of the I signal to the output terminal 5004.

The signal processing device 2 converts in the analog-to-digital converter 22 the X signal, the Y signal, the Z signal, the I signal, and the Q signal that are signals generated by the triaxial acceleration sensor 12A and the quadrature detection circuit 5A from analog signals into digital signals, and further temporarily or semi-permanently stores the signals converted into the digital signals in the storage device 23.

Next, connection of each terminal will be described. The terminal 3001 of the signal generation unit 3 and the terminal 4201 of the divider circuit 42 are connected, the terminal 4202 of the divider circuit 42 and the terminal 4101 of the divider circuit 41 are connected, the terminal 4103 of the divider circuit 41 and the terminal 1111 of the antenna 111 are connected, the terminal 4102 of the divider circuit 41 and the terminal 5001 of the quadrature detection circuit 5A are connected, and the terminal 4203 of the divider circuit 42 and the terminal 5003 of the quadrature detection circuit 5A are connected.

Next, an operation of the vibration detection device D2 will be described. A high frequency signal is output from the output terminal 3001 of the signal generation unit 3, and is input to the terminal 4201 of the divider circuit 42. The input signal is divided into two by the divider circuit 42, and the divided signals are output from the terminal 4202 and the terminal 4103. A signal output from the terminal 4202 of the divider circuit 42 is input to the terminal 4101 of the divider circuit 41, and is output from the terminal 4103 of the divider circuit 41. The output signal is input to the terminal 1111 of the antenna 111, and is radiated as a radio wave from the antenna 111. The radio wave radiated from the antenna 111 is reflected by the detection target or an object that exists next to the detection target, and input as a signal of a reflected wave to the antenna 111. The reflected signal input to the antenna 111 is output from the terminal 1111, and is input to the terminal 4103 of the divider circuit 41. The reflected signal input to the terminal 4103 of the divider circuit 41 is output from the terminal 4102. The signal output from the terminal 4203 of the divider circuit 42 is input to the terminal 5003 of the quadrature detection circuit 5A, and the signal output from the terminal 4102 of the divider circuit 41 is input to the terminal 5001 of the quadrature detection circuit 5A, these signals are subjected to quadrature detection by the quadrature detection circuit 5A, and the I signal and the Q signal are output from the output terminal 5002 and the output terminal 5004 to the signal processing device 2.

Furthermore, the signal detected by the triaxial acceleration sensor 12A is output from the output terminals 1201, 1202, and 1203 of the triaxial acceleration sensor 12A to the signal processing device 2.

Furthermore, parts that can be mounted on a substrate and have each function described in the present embodiment constitute the vibration detection device D3, so that all functions can be implemented as the vibration detection device D3 on the substrate. For example, the antenna 111 can be formed of a copper foil pattern or can be constituted using parts that can be mounted on the substrate and have the functions of the triaxial acceleration sensor 12A, the signal generation unit 3, the divider circuits 41 and 42, the quadrature detection circuit 5A, and the signal processing device 2. At this time, a permittivity and a layer configuration of the substrate are design matters as appropriate, and any material and layer configuration may be used as long as the material or the layer configuration have the functions of the vibration detection device D3.

As described above, in the present embodiment, the vibration detection device D3 is configured using the quadrature detection circuit 5A as the detection circuit 5 in Embodiment 2, and the triaxial acceleration sensor 12A as the inertial sensor 12. The learning model 2B of the signal processing device 2 infers a peak of a waveform using all information of the I signal, the Q signal, the X signal, the Y signal, and the Z signal obtained by the radio wave sensor 11 and the triaxial acceleration sensor 12A included in the sensor unit 1, so that it is possible to complement accuracy deterioration due to data missing, and implement the vibration detection device D3 that can operate with a high S/N ratio in a compact and low cost manner.

### Embodiment 4

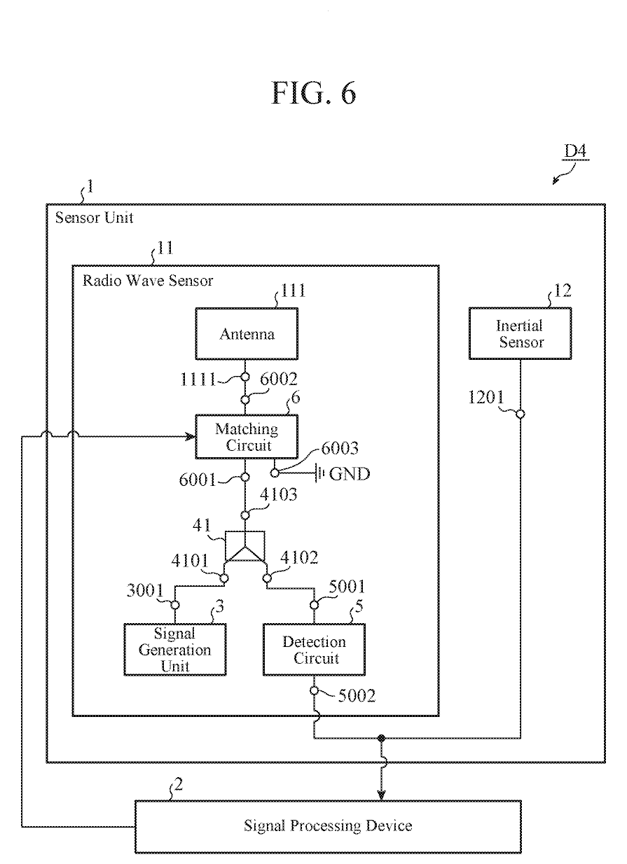

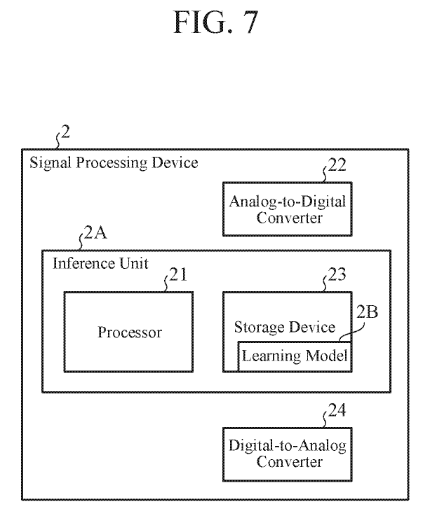

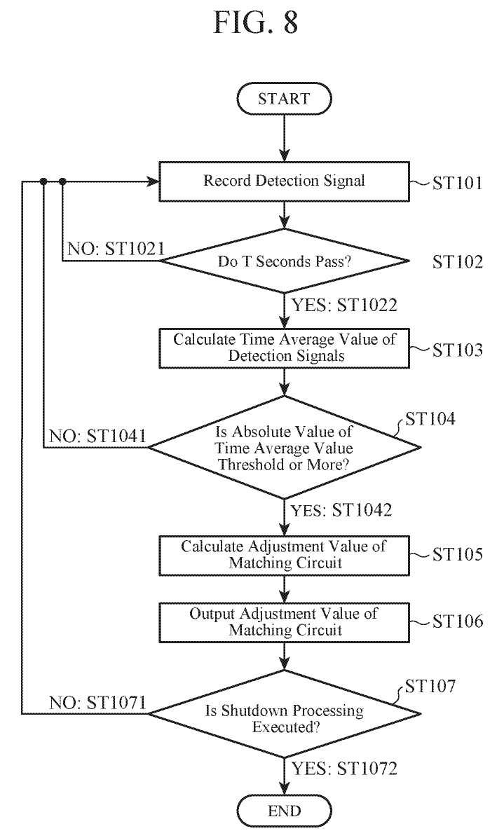

A configuration where a matching circuit that can be adaptively controlled is added between the divider circuit and the antenna in Embodiment 2 or 3 will be described in Embodiment 4. FIG. 6 is a configuration diagram illustrating a vibration detection device D4 according to the present embodiment. Although FIG. 6 illustrates a configuration where the matching circuit is added to Embodiment 2, the matching circuit may be added between the divider circuit and the antenna even in a case of Embodiment 3 in accordance with FIG. 6. FIG. 7 is a function diagram illustrating of a signal processing device according to the present embodiment. FIG. 8 is a flowchart of an adaptive control program of the matching circuit. Note that the same reference numerals in FIGS. 6 to 8 as those in FIG. 1 indicate identical or corresponding portions.

As illustrated in FIG. 6, the matching circuit 6 is disposed between the divider circuit 41 and the antenna 111. The matching circuit 6 is a circuit that includes three terminals 6001, 6002, and 6003, and matches a load impedance connected to the terminal 6002 with an impedance of a circuit connected to the terminal 6001, and can vary pass and reflection characteristics. Furthermore, the terminal 6003 is grounded to a GND.

The matching circuit 6 includes a variable element that can be adaptively controlled, and a variable capacitance diode, an air variable capacitor, or a poly variable capacitor may be used as the variable element.

Each terminal of the matching circuit 6 is connected as follows. The terminal 6001 is connected to the terminal 4103 of the divider circuit 41, the terminal 6002 is connected to the terminal 1111 of the antenna 111, and the terminal 6003 is grounded.

Furthermore, by adaptively changing a value of the variable element according to a control signal sent from the signal processing device 2, the matching circuit 6 can adjust a circuit constant depending on a change in load impedance connected to the matching circuit 6.

FIG. 7 is a configuration diagram of the signal processing device 2, and a digital-to-analog converter 24 that converts digital signals into analog signals is provided in addition to the components illustrated in FIG. 4.

The digital-to-analog converter 24 converts a digital signal that is a computation result of the processor 21 into an analog signal that the matching circuit 6 can accept. Note that, for the digital-to-analog converter 24, hardware that performs dedicated processing may be used or a program that performs the same processing may be used.

Next, an adjustment method of the matching circuit will be described with reference to a flowchart in FIG. 8. When powered on in accordance with a control program, the processor 21 of the vibration detection device D4 stores in the storage device 23 a detection signal (referred to as an “Id signal” for convenience of description) of the detection circuit 5 or the quadrature detection circuit 5A converted into a digital signal and the value of the variable element of the matching circuit 6 (step ST101). Hereinafter, an operation in each step is performed by the processor 21 in accordance with the control program.

In a case where certain measurement time T seconds do not pass (NO: step ST102), recording signals continues, and, in a case where the T seconds pass (YES: step ST103), an absolute value of a time average value of Id signals for the T seconds stored in the storage device 23 is calculated (step ST103). Note that, when shutdown processing is not executed even while calculation of a time average or other processing is executed, recording signals continues.

In a case where the absolute value of the time average of the values of the Id signals goes below a certain threshold Δ (NO: ST1041), processing returns to ST101, and, in a case where this absolute value is the certain threshold Δ or more (YES: ST1042), an adjustment value (the value of the variable element) is calculated to match a load impedance connected to the terminal 6002 of the matching circuit 6 with an impedance of a circuit connected to the terminal 6001 (step ST105). Furthermore, the calculated adjustment value is output to the matching circuit 6 (step ST106).

Next, whether or not shutdown processing is executed with respect to the processor 21 is determined, and, in a case where the shutdown processing is not executed (NO: step ST1071), processing returns to ST101, and, in a case where the shutdown processing is executed (YES: ST1072), the processor 21 executes the shutdown processing, and ends the adaptive control processing in FIG. 8.

As described above, by disposing the matching circuit 6 that can be adaptively controlled between the antenna 111 and the divider circuit 41, it is possible to implement the vibration detection device D4 in which a signal is acquired by the antenna 111 has a high S/N ratio.

### Embodiment 5

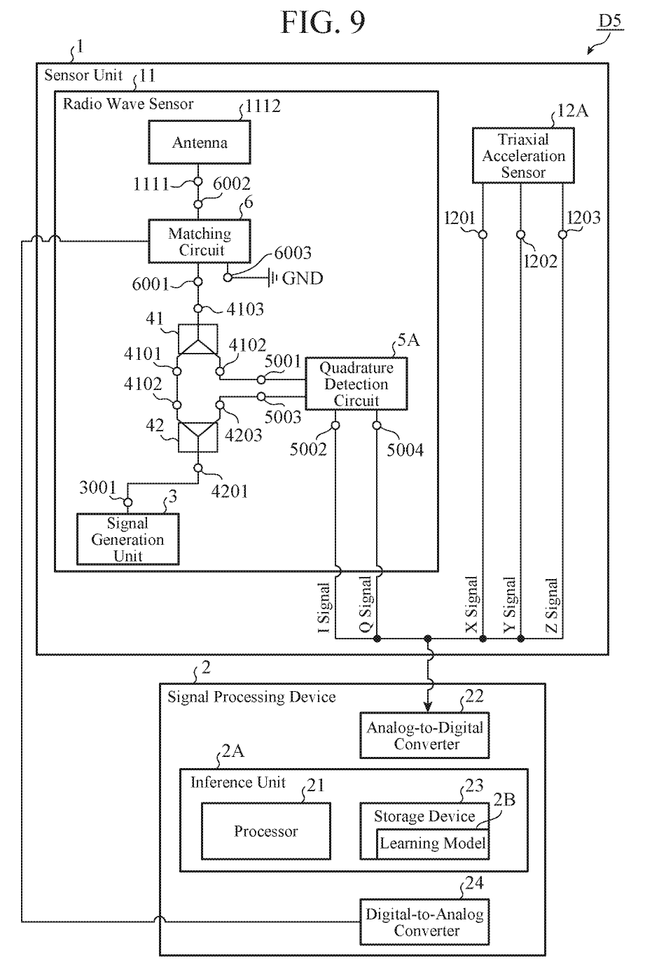

A measurement method in a case where a Conditional Variational AutoEncoder (CVAE) is implemented in machine learning in Embodiments 1 to 4 will be described in Embodiment 5. A method of acquiring a heartbeat waveform will be described as an example of the measurement method. FIG. 9 is a configuration diagram of a vibration detection device D5 according to Embodiment 5.

The vibration detection device D5 includes the loop antenna 1112 in FIG. 3A in the sensor unit in FIG. 5. Furthermore, as illustrated in FIG. 9, the vibration detection device D5 employs a configuration including the matching circuit 6 in FIG. 6, and including the signal processing device 2 in FIG. 7 that performs adaptive control processing on the matching circuit in FIG. 8.

The loop antenna 1112 described in the present embodiment has two loops, and the length of one side of the rectangular shape of the outermost shape surrounding the loop antenna 1112 is the magnitude of λ/100 of the use frequency. Note that the length described herein is one aspect, and, in a case where the antenna is a small antenna, and the length of the outermost shape is λ/10 or less of the use frequency, details of types, shapes, sizes, and the like are not limited, and various aspects may be selected according to a detection target type or the regulations of the Radio Act.

As for the vibration detection device D5 employing the configuration illustrated in FIG. 9, by forming the antenna 1112 with the copper foil pattern and using surface mounting parts as the signal generation unit 3 and other circuits, it is possible to form the vibration detection device D5 on one substrate.

The antenna 1112 patterned with the copper foil in the vibration detection device D5 is disposed at a position near a body surface near a heart without contacting the body surface. For example, the vibration detection device D5 is disposed on the clothes.

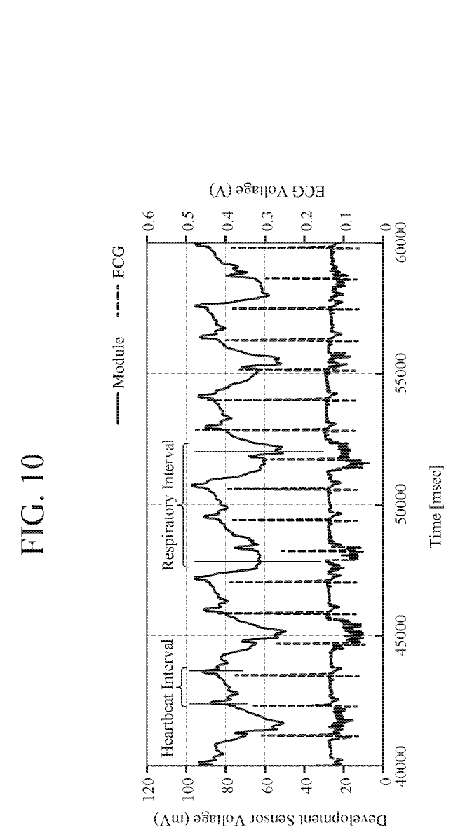

FIG. 10 illustrates an example of a waveform (heartbeat/respiratory waveforms) acquired by the vibration detection device D5 disposed on the clothes in a case where the radio wave sensor 11 including the matching circuit 6 and an algorithm (matching circuit adjustment algorithm) related to adaptive control processing of the matching circuit in FIG. 8 are used. In FIG. 10, a waveform indicated by “Module” indicates a waveform acquired by the vibration detection device D5. A “development sensor” in FIG. 10 means the vibration detection device D5. At this time, an electrocardiogram waveform is simultaneously acquired by synchronizing the waveform acquired by the vibration detection device D5 and a time. In FIG. 10, a waveform shown by an “ECG” (Electrocardiogram) indicates a waveform of an unillustrated electrocardiograph. By adaptively controlling the matching circuit using the matching circuit adjustment algorithm, it is possible to improve an S/N ratio of a signal acquired by the radio wave sensor 11, and acquire a waveform having a peak synchronized with a peak timing of the electrocardiogram waveform that is a correct answer value. Although a VHF band (30 to 300 MHz) has been used as a frequency of a radio wave radiated from the radio wave sensor 11, this frequency is merely an example, and other frequencies may be used.

Next, an example of a method of accurately inferring a peak of a heartbeat using machine learning will be described. The radio wave sensor 11 and the triaxial acceleration sensor 12A constituting the vibration detection device D5 are sensors that have different signal acquisition principles, yet can detect vibration caused by a motion of a detection target. Five items of time-series data of the I signal, the Q signal, the X signal, the Y signal, and the Z signal can be acquired from these two sensors.

The CVAE is a model that can capture a difference in distribution per class at a conditional probability by learning a data set with a class label on the basis of a Variational AutoEncoder (VAE) that is a deep generative model of an autoencoder network structure configured as a probabilistic graphical model with a latent variable. In the present embodiment, the class label refers to information of a correct answer value in other words. Note that the CVAE is merely an example of the learning model 2B, and the learning model 2B is not limited to the CVAE.

The I signal, the Q signal, the X signal, the Y signal, and the Z signal are input values, and an electrocardiogram waveform is given as a correct answer signal to generate class label data (learning data) in which an acquired signal and the correct answer signal are corresponded. The class label data (learning data) obtained here is stored in the storage device 23 of the signal processing device 2. At a time of inference, acquired data of the I signal, the Q signal, the X signal, the Y signal, and the Z signal is input to the learning model 2B to infer the electrocardiogram waveform. When a peak of a heartbeat waveform is detected, a window function of a triangle type or the like is prepared, and peaks and other components are classified and learned to further improve inference accuracy. The difference in distribution per class is captured at the conditional probability to perform inference, so that noise is reduced.

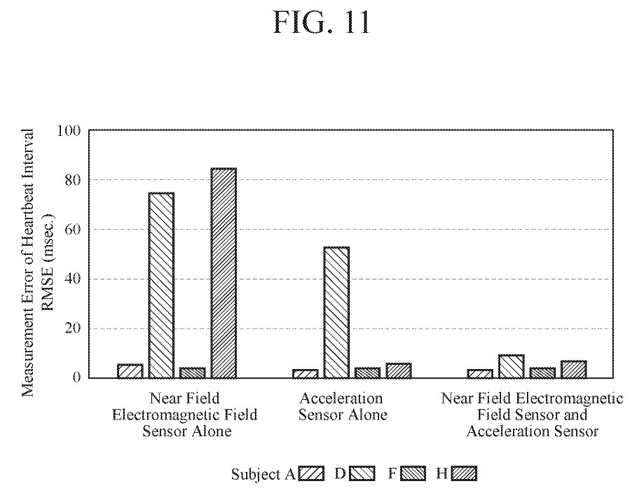

FIG. 11 is a view illustrating a comparison result obtained by comparing per subject a value of a Root Mean Square Error (RMSE) between a peak interval of a heartbeat waveform inferred using the CVAE per data used for learning, and a peak interval of an electrocardiogram waveform that is a correct answer value. There are four subjects A, D, F, and H.

FIG. 11 illustrates 1) a case where training is performed using the I signal and the Q signal obtained by a radio wave sensor (legend: a near field electromagnetic sensor alone), and an electrocardiogram waveform, 2) a case where training is performed using the X signal, the Y signal, and the Z signal obtained by a triaxial acceleration sensor (legend: an acceleration sensor alone), and an electrocardiogram waveform, and 3) a case where training is performed using the I signal, the Q signal, the X signal, the Y signal, and the Z signal obtained by a sensor configuration (legend: the near field electromagnetic sensor and the acceleration sensor) in the present embodiment, and an electrocardiogram waveform.

As is clear from FIG. 11, an error is reduced by performing training using all data obtained by the sensor configuration described in the present embodiment instead of performing training using data obtained by a single sensor. Accordingly, according to the present embodiment, it is possible to obtain an effect of improving inference accuracy.

As described above, by performing machine learning using all signals obtained by the configuration that uses the radio wave sensor and the triaxial acceleration sensor, and inferring a peak of a waveform, it is possible to obtain a vibration detection device that can complement accuracy deterioration due to data missing and operate with a high S/N ratio.

### <Supplementary Notes>

Some aspects of the above-described various embodiments will be summarized as follows.

### (Supplementary Note 1)

A vibration detection device according to Supplementary Note 1 includes: a radio wave sensor (11) to detect a change in near field electromagnetic field; an inertial sensor (12); and a signal processing device (2) to input waveform data of the radio wave sensor and waveform data of the inertial sensor to a learning model, and acquire from the learning model an inference result of vibration inferred by the learning model.

### (Supplementary Note 2)

A vibration detection device according to Supplementary Note 2 is the vibration detection device described in Supplementary Note 1, and the radio wave sensor, the inertial sensor, and the signal processing device are disposed on an identical substrate.

### (Supplementary Note 3)

A vibration detection device according to Supplementary Note 3 is the vibration detection device described in Supplementary Note 1 or 2, the radio wave sensor detects the change in the electromagnetic field caused by a motion of a lung of a person that is a detection target, the inertial sensor detects local vibration caused by the motion of the lung, and the signal processing device infers a peak of respiration of the person as the vibration.

### (Supplementary Note 4)

A vibration detection device according to Supplementary Note 4 is the vibration detection device described in Supplementary Note 1 or 2, the radio wave sensor detects the change in the electromagnetic field caused by a motion of a heart of a person that is a detection target, the inertial sensor detects local vibration caused by the motion of the heart, and the signal processing device infers a peak of respiration of the person as the vibration.

### (Supplementary Note 5)

A vibration detection device according to Supplementary Note 4 is the vibration detection device described in any one of Supplementary Notes 1 to 4, and, as illustrated in FIG. 2, the radio wave sensor includes a signal generation unit (3) to generate a high frequency signal, an antenna (111) to transmit the generated high frequency signal, a detection circuit (5) to detect an input signal, generate a detection signal, and output the generated detection signal, a divider circuit (41) to output a signal input to a first terminal (4101) from a third terminal (4103), and divide and output a signal input to the third terminal (4103) to the first terminal (4101) and a second terminal (4102), the divider circuit (41) including the first terminal (4101), the second terminal (4102), and the third terminal (4103), and the first terminal being connected to the signal generation unit, the second terminal being connected to the detection circuit, and the third terminal being connected to the antenna.

### (Supplementary Note 6)

A vibration detection device according to Supplementary Note 6 is the vibration detection device described in any one of Supplementary Notes 1 to 4, as illustrated in FIG. 6, the radio wave sensor includes a signal generation unit (3) to generate a high frequency signal, an antenna (111) to transmit the generated high frequency signal, a detection circuit (5) to detect an input signal, generate a detection signal, and output the generated detection signal, a divider circuit (41) to output a signal input to a first terminal (4101) from a third terminal (4103), and divide and output a signal input to the third terminal (4103) to the first terminal (4101) and a second terminal (4102), the divider circuit (41) including the first terminal (4101), the second terminal (4102), and the third terminal (4103), and the first terminal being connected to the signal generation unit, and the second terminal being connected to the detection circuit, and a matching circuit (6) including a fourth terminal (6001) connected to the third terminal of the divider circuit, and a fifth terminal (6002) connected to the antenna, and including a variable element, and the signal processing device outputs a control signal for controlling an adjustment value of the variable element to the matching circuit to match an impedance of the fourth terminal of the matching circuit and an impedance of the fifth terminal on the basis of a value of the detection signal output from the detection circuit.

### (Supplementary Note 7)

A vibration detection device according to Supplementary Note 7 is the vibration detection device described in any one of Supplementary Notes 1 to 4, as illustrated in FIG. 9, the radio wave sensor includes a signal generation unit (3) to generate a high frequency signal, an antenna (111) to transmit the generated high frequency signal, a detection circuit (5) to detect an input signal, generate a detection signal, and output the generated detection signal, a first divider circuit (41) to output a signal input to a first terminal (4101) from a third terminal (4103), and divide and output a signal input to the third terminal (4103) to the first terminal (4101) and a second terminal (4102), the first divider circuit (41) including the first terminal (4101), the second terminal (4102), and the third terminal (4103), and the second terminal being connected to the detection circuit, a second divider circuit (42) to output a signal input to a fifth terminal (4202) from a fourth terminal (4201), and divide and output a signal input to the fourth terminal (4201) to the fifth terminal (4202) and a sixth terminal (4203), the second divider circuit (42) including the fourth terminal (4201), the fifth terminal (4202), and the sixth terminal (4203), and the fourth terminal being connected to the signal generation unit, the fifth terminal being connected to the first terminal of the first detection circuit, and the sixth terminal being connected to the detection circuit, and a matching circuit (6) including a seventh terminal (6001) connected to the third terminal of the first divider circuit, and an eighth terminal (6002) connected to the antenna, and including a variable element, the detection circuit (quadrature detection circuit 5A) performs quadrature detection on a signal input from the first divider circuit and a signal input from the second divider circuit as local signals to generate an IQ signal, and outputs the generated IQ signal as the detection signal, and the signal processing device outputs a control signal for controlling an adjustment value of the variable element to the matching circuit to match an impedance of the seventh terminal of the matching circuit and an impedance of the eighth terminal on the basis of a value of the detection signal output from the detection circuit.

### (Supplementary Note 8)

A vibration detection device according to Supplementary Note 8 is the vibration detection device described in any one of Supplementary Notes 5 to 7, and, in a case where the antenna has a rectangular shape, and a wavelength of the high frequency signal is λ, a length of one side of the rectangular shape of an outermost shape of the antenna is λ/10 or less.

### (Supplementary Note 9)

A vibration detection method according to Supplementary Note 9 is a vibration detection method of a vibration detection device that includes a radio wave sensor (11), an inertial sensor (12), and a signal processing device (2), and includes: a step of, by the radio wave sensor, detecting a change in near field electromagnetic field (ST1); a step of, by the inertial sensor, detecting local vibration of a detection target (ST2); and a step of, by the signal processing device, inputting waveform data of the radio wave sensor and waveform data of the inertial sensor to a learning model, and acquiring from the learning model an inference result of vibration inferred by the learning model (ST3).

Note that the embodiments can be combined, and each embodiment can be modified or omitted as appropriate.

### INDUSTRIAL APPLICABILITY

The vibration detection device according to the present disclosure can be used as a device for acquiring, for example, vital information such as a heart rate of a person.

### REFERENCE SIGNS LIST

1: Sensor unit, 2: Signal processing device, 2A: Inference unit, 2B: Learning model, 3: Signal generation unit, 5: Detection circuit, 5A: Quadrature detection circuit, 6: Matching circuit, 11: Radio wave sensor, 12: Inertial sensor, 12A: Triaxial acceleration sensor, 21: Processor, 22: Analog-to-digital converter, 23: Storage device, 24: Digital-to-analog converter, 41: Divider circuit, 42: Divider circuit, 111: Antenna, 1112: Loop antenna, 1113: Meander dipole antenna, D1 to D5: Vibration detection device

1. A vibration detection device comprising:
a radio wave sensor to detect a change in near field electromagnetic field;
an inertial sensor; and
a signal processor to input waveform data of the radio wave sensor and waveform data of the inertial sensor to a learning model, and acquire from the learning model an inference result of vibration inferred by the learning model,
wherein
the radio wave sensor detects the change in the electromagnetic field caused by a motion of a lung of a person that is a detection target,
the inertial sensor detects local vibration caused by the motion of the lung, and
the signal processor infers a peak of respiration of the person as the vibration using all information obtained by the radio wave sensor and the inertial sensor.

2. A vibration detection device comprising:
a radio wave sensor to detect a change in near field electromagnetic field;
an inertial sensor; and
a signal processor to input waveform data of the radio wave sensor and waveform data of the inertial sensor to a learning model, and acquire from the learning model an inference result of vibration inferred by the learning model, wherein
the radio wave sensor detects the change in the electromagnetic field caused by a motion of a heart of a person that is a detection target,
the inertial sensor detects local vibration caused by the motion of the heart, and
the signal processor infers a peak of heartbeat of the person as the vibration using all information obtained by the radio wave sensor and the inertial sensor.

3. The vibration detection device according to claim 1, wherein the radio wave sensor, the inertial sensor, and the signal processor are disposed on an identical substrate.

4. The vibration detection device according to claim 1, wherein the radio wave sensor includes
a signal generator to generate a high frequency signal,
an antenna to transmit the generated high frequency signal,
a detection circuit to detect an input signal, generate a detection signal, and output the generated detection signal, and
a divider circuit to output a signal input to a first terminal from a third terminal, and divide and output a signal input to the third terminal to the first terminal and a second terminal, the divider circuit including the first terminal, the second terminal, and the third terminal, and the first terminal being connected to the signal generator, the second terminal being connected to the detection circuit, and the third terminal being connected to the antenna.

5. The vibration detection device according to claim 1, wherein
the radio wave sensor includes
a signal generator to generate a high frequency signal,
an antenna to transmit the generated high frequency signal,
a detection circuit to detect an input signal, generate a detection signal, and output the generated detection signal,
a divider circuit to output a signal input to a first terminal from a third terminal, and divide and output a signal input to the third terminal to the first terminal and a second terminal, the divider circuit including the first terminal, the second terminal, and the third terminal, and the first terminal being connected to the signal generator, and the second terminal being connected to the detection circuit, and
a matching circuit including a fourth terminal connected to the third terminal of the divider circuit, and a fifth terminal connected to the antenna, and including a variable element, and
the signal processor outputs a control signal for controlling an adjustment value of the variable element to the matching circuit to match an impedance of the fourth terminal of the matching circuit and an impedance of the fifth terminal on a basis of a value of the detection signal output from the detection circuit.

6. The vibration detection device according to claim 1, wherein
the radio wave sensor includes
a signal generator to generate a high frequency signal,
an antenna to transmit the generated high frequency signal,
a detection circuit to detect an input signal, generate a detection signal, and output the generated detection signal,
a first divider circuit to output a signal input to a first terminal from a third terminal, and divide and output a signal input to the third terminal to the first terminal and a second terminal, the first divider circuit including the first terminal, the second terminal, and the third terminal, and the second terminal being connected to the detection circuit,
a second divider circuit to output a signal input to a fifth terminal from a fourth terminal, and divide and output a signal input to the fourth terminal to the fifth terminal and a sixth terminal, the second divider circuit including the fourth terminal, the fifth terminal, and the sixth terminal, and the fourth terminal being connected to the signal generator, the fifth terminal being connected to the first terminal of the first detection circuit, and the sixth terminal being connected to the detection circuit, and
a matching circuit including a seventh terminal connected to the third terminal of the first divider circuit, and an eighth terminal connected to the antenna, and including a variable element,
the detection circuit performs quadrature detection on a signal input from the first divider circuit and a signal input from the second divider circuit as local signals to generate an IQ signal, and outputs the generated IQ signal as the detection signal, and
the signal processor outputs a control signal for controlling an adjustment value of the variable element to the matching circuit to match an impedance of the seventh terminal of the matching circuit and an impedance of the eighth terminal on a basis of a value of the detection signal output from the detection circuit.

7. The vibration detection device according to claim 4, wherein, in a case where the antenna has a rectangular shape, and a wavelength of the high frequency signal is λ, a length of one side of the rectangular shape of an outermost shape of the antenna is λ/10 or less.

8. The vibration detection device according to claim 5, wherein, in a case where the antenna has a rectangular shape, and a wavelength of the high frequency signal is λ, a length of one side of the rectangular shape of an outermost shape of the antenna is λ/10 or less.

9. A vibration detection method of a vibration detection device that comprises a radio wave sensor, an inertial sensor, and a signal processor, the vibration detection method comprising:
by the radio wave sensor, detecting a change in near field electromagnetic field and detecting a change of an electromagnetic field by a move of lung of a person that is a detection target;
by the inertial sensor, detecting local vibration of a detection target and detecting local vibration caused by the move of the lung; and
by the signal processor, inputting waveform data of the radio wave sensor and waveform data of the inertial sensor to a learning model, and acquiring from the learning model an inference result of vibration inferred by the learning model,
wherein the signal processor infers a peak of respiration of the person as the vibration using all information obtained by the radio wave sensor and the inertial sensor.

10. The vibration detection device according to claim 2, wherein the radio wave sensor, the inertial sensor, and the signal processor are disposed on an identical substrate.

11. The vibration detection device according to claim 2, wherein the radio wave sensor includes
a signal generator to generate a high frequency signal,
an antenna to transmit the generated high frequency signal,
a detection circuit to detect an input signal, generate a detection signal, and output the generated detection signal, and
a divider circuit to output a signal input to a first terminal from a third terminal, and divide and output a signal input to the third terminal to the first terminal and a second terminal, the divider circuit including the first terminal, the second terminal, and the third terminal, and the first terminal being connected to the signal generator, the second terminal being connected to the detection circuit, and the third terminal being connected to the antenna.

12. The vibration detection device according to claim 2, wherein
the radio wave sensor includes
a signal generator to generate a high frequency signal,
an antenna to transmit the generated high frequency signal,
a detection circuit to detect an input signal, generate a detection signal, and output the generated detection signal,
a divider circuit to output a signal input to a first terminal from a third terminal, and divide and output a signal input to the third terminal to the first terminal and a second terminal, the divider circuit including the first terminal, the second terminal, and the third terminal, and the first terminal being connected to the signal generator, and the second terminal being connected to the detection circuit, and
a matching circuit including a fourth terminal connected to the third terminal of the divider circuit, and a fifth terminal connected to the antenna, and including a variable element, and
the signal processor outputs a control signal for controlling an adjustment value of the variable element to the matching circuit to match an impedance of the fourth terminal of the matching circuit and an impedance of the fifth terminal on a basis of a value of the detection signal output from the detection circuit.

13. The vibration detection device according to claim 2, wherein
the radio wave sensor includes
a signal generator to generate a high frequency signal,
an antenna to transmit the generated high frequency signal,
a detection circuit to detect an input signal, generate a detection signal, and output the generated detection signal,
a first divider circuit to output a signal input to a first terminal from a third terminal, and divide and output a signal input to the third terminal to the first terminal and a second terminal, the first divider circuit including the first terminal, the second terminal, and the third terminal, and the second terminal being connected to the detection circuit,
a second divider circuit to output a signal input to a fifth terminal from a fourth terminal, and divide and output a signal input to the fourth terminal to the fifth terminal and a sixth terminal, the second divider circuit including the fourth terminal, the fifth terminal, and the sixth terminal, and the fourth terminal being connected to the signal generator, the fifth terminal being connected to the first terminal of the first detection circuit, and the sixth terminal being connected to the detection circuit, and
a matching circuit including a seventh terminal connected to the third terminal of the first divider circuit, and an eighth terminal connected to the antenna, and including a variable element,
the detection circuit performs quadrature detection on a signal input from the first divider circuit and a signal input from the second divider circuit as local signals to generate an IQ signal, and outputs the generated IQ signal as the detection signal, and
the signal processor outputs a control signal for controlling an adjustment value of the variable element to the matching circuit to match an impedance of the seventh terminal of the matching circuit and an impedance of the eighth terminal on a basis of a value of the detection signal output from the detection circuit.
